(19) United States
(12) Patent Application Publication
(10) Pub. No.: US 2008/0108543 A1
(43) Pub. Date: May 8, 2008

(54) Outdoor fragrance composition

(76) Inventors: Michael B. North, Greenacres, FL (US); Juanita North, Greenacres, FL (US)

Correspondence Address: ROBERT M. DOWNEY, P.A., 6751 N. FEDERAL HWY., SUITE 300, BOCA RATON, FL, 33487, US

(21) Appl. No.: 11/593,951

(22) Filed: Nov. 6, 2006

(51) Int. Cl. A61L 9/013

(52) U.S. Cl. 512/5

(57) ABSTRACT

An outdoor fragrance composition for sprinkle application to gardens, plant and flower beds, lawns, pots and planters. The composition provides a long lasting pleasant scent that smells natural. The composition includes saw dust, dye, fragrance oil, and water. The fragrance oil may be any of a variety of fragrances and is preferably selected from a variety of floral fragrances. The dye provides a soil color, or other color that blends in with the natural ground application area. In an alternative embodiment, cracked wheat is substituted for saw dust.

### BACKGROUND OF THE INVENTION

1. Field of the Invention

The present invention is directed to air fragrance compositions and, more particularly, to an outdoor fragrance composition for sprinkle application to gardens, plant and flower beds, lawns, pots and planters to provide a long lasting pleasant and natural scent.

2. Discussion of the Related Art

Air fragrances have typically been intended for indoor use to provide a pleasant odor. In some instances, indoor air fresheners are used to mask unpleasant odors, such as from pets, mildew or cooking. Indoor fragrances are also used to enhance the indoor environment by providing a pleasant scent. Air fresheners for indoor use are provided in a variety of forms including sprays, solid substances, and electric outlet plug-in devices that heat solid or oil fragrances. Indoor fragrances have also been incorporated into various powder substances, such as baking soda, for sprinkle application to carpets. Additionally, fragrances are often added to candles for indoor and outdoor use.

Despite the numerous developments of fragrance compositions, most of which have been intended primarily for indoor use, there still remains a need for a practical and long lasting fragrance composition that can be used outdoors, such as in a garden or plant and flower beds, to provide a natural floral aroma.

### SUMMARY OF THE INVENTION

The present invention is directed to an outdoor fragrance composition for sprinkle application to gardens, plant and flower beds, lawns, pots and planters. The composition provides a long lasting scent that is pleasant and natural. The composition includes saw dust, dye, fragrance oil, and water. The fragrance oil may be any of a variety of fragrances and is preferably selected from a variety of floral fragrances. The dye provides a soil color, or other color that blends in with the natural area of application. In an alternative embodiment, the composition includes cracked wheat, color dye, fragrance oil and water.

### OBJECTS AND ADVANTAGES OF THE INVENTION

Considering the foregoing, it is a primary object of the present invention to provide an outdoor fragrance composition for sprinkle application to gardens, plant and flower beds, lawns, pots and planters to provide a long lasting pleasant scent in an outdoor environment.

It is a further object of the present invention to provide an outdoor fragrance composition for sprinkle application to gardens, plant and flower beds, lawns, pots and planters, and wherein the composition blends with the surrounding environment, such as top soil so that the composition is not visible after application.

It is a further object of the present invention to provide an outdoor fragrance composition that provides a long lasting natural scent.

It is a further object of the present invention to provide an outdoor fragrance composition that is easily applied to a garden, lawn, plant and/or flower beds by sprinkle application, and wherein the fragrance composition provides a long lasting and pleasant scent that emanates the surrounding atmosphere.

It is a further object of the present invention to provide an outdoor fragrance composition that is easy and inexpensive to manufacture and which avoids use of harmful chemicals.

These and other objects and advantages of the invention are more readily apparent with reference to the entire specification.

### DETAILED DESCRIPTION OF THE PREFERRED EMBODIMENT

The fragrance composition of the present invention is intended primarily for outdoor use. The composition is applied by sprinkling to the applied area, such as to the soil in gardens, plant and flower beds, as well as in plant pots and planters. The fragrance composition can also be applied by sprinkle application to lawns and other outdoor areas.

In a preferred embodiment of the present invention, the outdoor fragrance composition comprises a blend of saw dust, dye, and fragrance oil mix with a small amount of water. The fragrance oil is selected form any of a variety of fragrances that are commonly available. The saw dust is preferably ground to a texture that is similar to fine top soil. In an alternative embodiment, cracked wheat is used instead of saw dust. The dye is added to change the natural color of the saw dust to a color that blends with the applied environment, such as top soil. For instances, when applying to gardens and flower beds, the dye provides a soil color that closes matches the color of the soil in the garden or flower bed.

An example of the composition is as follows:

EXAMPLE 1

| Ingredient | Amount (% by weight) |
|---|---|
| Saw Dust | 79% |
| Fragrance Oil | 13% |
| Dye | 3% |
| Water | 5% |

In a preferred embodiment saw dust or cracked wheat is present in the composition in an amount ranging between 40% and 90% by weight. The dye may be of any environmentally approved color and formulation is present in the composition in an amount ranging between 0.5% and 5.0% by weight. Fragrance oil is present in the composition in the amount ranging between 3% and 25% by weight. Water is added to the composition to promote blending of the ingredients and penetration of the dye color into the saw dust. Water also helps to promote dispersion of the fragrance oil within the composition and release of the fragrance when applied to the ground surface area. Water is present in the composition in the amount ranging between 1% and 8% by weight.

As previously mentioned, the fragrance oil may be selected from any of a variety of available fragrances. In a preferred embodiment, the fragrance is a floral aroma to provide a natural garden scent. Examples of fragrances that are contemplated for use in the composition include, but are not limited to, the following:
Jasmine
Orange Blossom
Sweet Pea
Christmas Spice
Gardenia
Rose
Floral Blossom
Lavender

While present invention has been described in accordance with a preferred and practical embodiment thereof, it is recognized that departures from the instant disclosure are fully contemplated within the spirit and scope of the present invention as defined in the following claims and under the doctrine of equivalence.

What is claimed is:

1. An outdoor fragrance composition comprising:
saw dust;
fragrance oil;
dye color; and
water.

2. An outdoor fragrance composition comprising:
saw dust in an amount ranging between 40% and 90% by weight of the composition;
fragrance oil in an amount ranging 3% and 25% by weight of the composition;
dye color in an amount ranging between 0.5% and 5.0% by weight of the composition; and
water in an amount ranging between 1% and 8% by weight of the composition.

3. An outdoor fragrance composition comprising:
cracked wheat in an amount ranging between 40% and 90% by weight of the composition;
fragrance oil in an amount ranging 3% and 25% by weight of the composition;
dye color in an amount ranging between 0.5% and 5.0% by weight of the composition; and
water in an amount ranging between 1% and 8% by weight of the composition.
